(19) United States
(12) Patent Application Publication
(10) Pub. No.: US 2015/0183743 A1
(43) Pub. Date: Jul. 2, 2015

(54) COMPOUND AND USE OF COMPOUND TO PREPARE A RADIOLABELLED COMPOUND

(71) Applicant: STICHTING VU-VUMC, Amsterdam (NL)

(72) Inventors: Johannes Antonius Maria Christiaans, Amsterdam (NL); Albert Dirk Windhorst, Amsterdam (NL); Athanasios Metaxas, Amsterdam (NL); Pieter Jacob Klein, Amsterdam (NL); Esther Johanna Maria Kooijman, Amsterdam (NL); Bart Nicolaas Maria Van Berckel, Amsterdam (NL)

(21) Appl. No.: 14/412,329

(22) PCT Filed: Jul. 4, 2013
(86) PCT No.: PCT/NL2013/050497

§ 371 Date: Dec. 31, 2014

(30) Foreign Application Priority Data
Jul. 5, 2012 (NL) 2009131

(51) Int. Cl. C07D 213/74

(52) U.S. Cl. CPC C07D 213/74

(57) ABSTRACT

The invention is directed to a compound according to formula (1) wherein R² is fluorine, X is nitrogen or carbon and R³ is an organic group. The invention is also directed to the use of this compound as a precursor for the preparation of a ¹¹C or ¹⁸F-radio labelled compound. The radio labelled compound may be used in an in vivo diagnostic or imaging method.

The invention is directed to the following compound

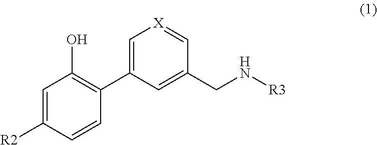

wherein R² is fluorine, X is nitrogen or carbon and R³ is an organic group.

Applicants have found that this new class of compounds may advantageously be used to prepare compounds which have an improved affinity to the NR2B binding site of the NMDAR complex. It has been speculated that selective antagonists of the NR2B subtype might provide a cleaner side effect profile compared to antagonists of the glycine binding site or blockers of the ion channel. Neuropharmacology 1999, 38, 611-623. More especially it is found that this new class of the compounds allow a quick synthesis of radio labelled compounds thus allowing an in vivo diagnosis or imaging of NMDA related disease.

Current Medicinal Chemistry 2004, 389-396 describes the increasing interest of antagonists for the NR2B binding site as a therapeutic target in a wide range of CNS pathologies, including acute and chronic pain, stroke and head trauma, drug-induced dyskinesias, and dementias.

Bioorganic Medicinal Chemistry Letters 2011, 3399-3403 describes a wide range of possible 2,6-disubstituted aromatic and heteroaromatic compounds which according to this publication may be useful as a pharmaceutical active compound for treatment in depression.

R³ in formula (1) is an organic group and preferably a branched or unbranched C₁-C₆ alkyl, C₃-C₇ cycloalkyl or a methyl C₃-C₇ cycloalkyl group. Examples of alkyl groups are methyl, ethyl or n-propyl, Examples of cycloalkyl groups are, C₃-C₇ cycloalkyl and preferably cyclopentyl, cyclobutyl. Examples of methyl cycloalkyl groups are —CH₂-cyclopropyl, —CH₂-cyclobutyl, —CH₂-cyclopentyl and preferably —CH₂-cyclopropyl.

X in formula (1) may be carbon or nitrogen. When X is carbon it will be understood that a hydrogen atom is attached to the carbon atom to create a phenyl ring. If X is carbon applicants found that a preferred compound may be prepared from this precursor compound which is a selective antagonist of the NR2B binding site of the N-methyl-D-aspartate (NMDA) receptor. This preferred compound is 1-cyclopropyl-N-((4′-fluoro-2′-methoxy-[1,1′-biphenyl]-3-yl)methyl)methanamine or a salt or solvate thereof.

A preferred group of compounds are compounds wherein X is nitrogen. Applicants found a group of compounds may be prepared from this compound having excellent selectivity towards the NR2B binding site as expressed in Ki.

The compounds according to formula (1) may be prepared by a known aryl ether cleavage reaction, for example a demethylation or other dealkylation reaction according to the below reaction scheme:

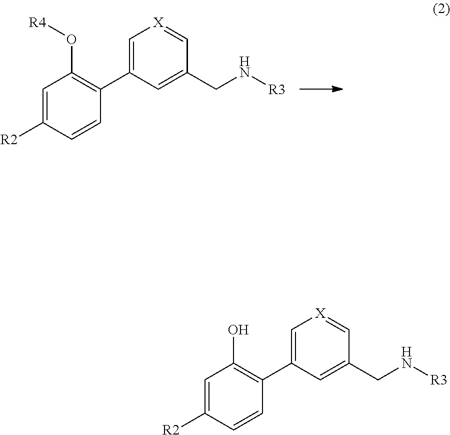

wherein R⁴ may be any group, preferably an alkyl group, for example a C₁₋₆ alkyl group, and more preferably methyl. This starting compound having a R⁴ group may be prepared according to the procedures described D. G. Brown et al., Bioorganic Medicinal Chemistry Letters (2011), 21, 3399-3403.

The invention is also directed to the use of the above compound according to formula (1) as a precursor to prepare a compound according to:

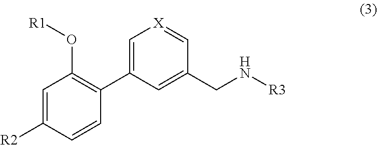

wherein R¹ is an optionally fluorinated alkyl group having 1 to 5 carbon atoms

When R¹ is not fluorinated it is preferred that R¹ is ethyl or methyl and preferably methyl. Preferably R¹ is a radio labelled substituent and even more preferably ¹¹C-methyl. R³ may be as described above. The preferred groups R³ are as above.

When R¹ is a fluorinated alkyl group having 1 to 5 carbon it is preferred that the alkyl group has from one to five fluorine substituents. Preferably the fluorinated alkyl group R¹ comprises from one to three carbon atoms. In the case that the compound is used to prepare a radio labelled compound it is preferred that the alkyl group R¹ is substituted with one fluorine atom and wherein the fluorine atom is a radio labelled ¹⁸F atom.

The precursor compound according to formula (1) is preferably used to prepare compounds according to formula (3) wherein R¹ is an optionally fluorinated alkyl group having 1 to 5 carbon atoms. The compounds may be radio labelled or non-radio labelled. The radio labelled compounds have a relatively short half life and are thus preferably prepared shortly before use in the above referred to in vivo diagnosis or imaging of NMDA related diseases. Suitably a non-radio labelled precursor compound according to formula (1) is synthesised which can, by means of a relatively simple synthesis, be reacted with a radio labelled compound to obtain the radio labelled compound as will be described below. These processes may also be used to prepare the non-radio labelled compounds using the equivalent non-radio labelled starting compounds.

The first preferred process is a process to prepare a radio labelled compound according to the following formula

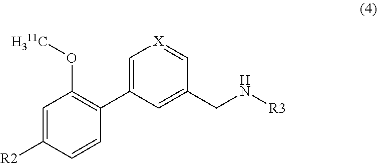

by alkylation reaction of a compound according to formula (1) as described above with a [¹¹C]CH₃L compound, wherein L is a leaving group. Leaving group L may be halogen, preferably iodo or another suitable leaving group such as alkyl or aryl sulfonate, such as, for example but not limited to, mesylate, triflate, tosylate or nosylate. The [¹¹C]CH₃L compound and, in the case that the leaving group L is iodine, the [¹¹C]CH₃L compound is prepared by known methods to a person skilled in the art.

The invention is especially directed to the novel precursor compound 2-(5-((cyclopentylamino)methyl)pyridin-3-yl)-5-fluorophenol and to its use as intermediate to prepare a ¹¹C labelled N-((5-(4-fluoro-2-¹¹C-methoxyphenyl)pyridin-3-yl)methyl)cyclopentanamine.

The second preferred process is a process to prepare a radio labelled compound according to the following formula

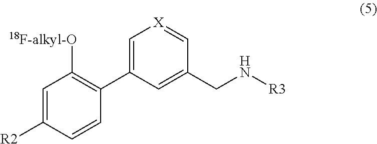

by alkylation reaction of a compound according to formula (¹) as described above with a ¹⁸F-alkyl-A compound wherein A is a leaving group according to the below reaction. Leaving group A may be halogen, preferably bromine or iodo or another suitable leaving group such as alkyl or aryl sulfonate, such as, for example but not limited to, mesylate, triflate, tosylate or nosylate.

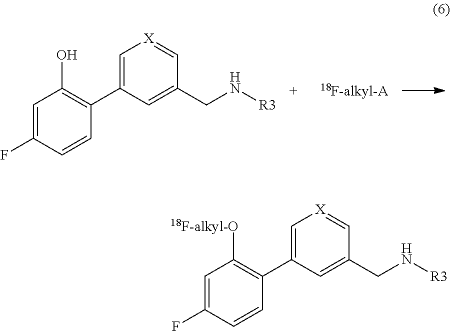

The precursor compound is preferably subjected to a nucleophilic fluorination, preferably carried out by heating or microwave irradiation of said precursor compound with [¹⁸F]fluoride complexed with a phase transfer catalyst such as (nBu)₄NHCO₃ or 4,7,13,16,21,24-Hexaoxa-1,10-diazabicyclo[8.8.8]hexacosane (Kryptofix[2.2.2]) in combination with or without a suitable base such as, but not limited to, potassium carbonate, potassium hydrogen carbonate, cesium carbonate in a suitable solvent such as, but not limited to, acetonitrile, N,N-dimethylformamide (DMF), dimethylsulfoxide (DMSO), sulfolane, ethanol, t-butanol or ionic liquids.

The ¹¹C-alkylation or ¹⁸F-alkylation reaction with the appropriate alkylhalide or alkylsulfonate is preferable carried out in a suitable solvent such as, but not limited to, acetone, acetonitrile, t-butanol, chloroform, dichloromethane, N,N-dimethylformamide (DMF), dimethylsulfoxide (DMSO), ethanol, isopropanol, methanol, propanol or tetrahydrofuran (THF) and in presence of a suitable base such as, but not limited to, cesium carbonate, potassium carbonate, potassium hydrogen carbonate, potassium hydroxide or sodium hydride, t-butylammonium hydroxide, triethylamine, diisopropylamine, diisopropylethylamine or dimethylaminopyridine and in presence or absence of a suitable catalyst such as, but not limited to, sodium iodide or potassium iodide.

The radio labelled compounds and non-radio labelled compounds as prepared according to the above may be purified according to those methods known to the person skilled in the art, for example by means of HPLC purification or Solid Phase Extraction (SPE). The HPLC purification is preferable carried out on a preparative HPLC column packed with reverse phase material such as, but not limited to, C18, C18-EPS or C8, a mobile phase consisting of a mixture of methanol, ethanol or acetonitrile mixed with water or water containing buffer such as, for example but not limited to, ammonium dihydrogen phosphate or an acid like phosphoric acid or trifluoracetic acid. The Solid Phase Extraction (SPE) is preferably performed on a Seppak® such as, for example but not limited to, C18, tC18, Silica or an Oasis Seppak®. The compound is preferably eluted from the Seppak® with a solvent suitable for injection in vivo, for example ethanol.

The above treated compounds may be formulated to a desired formulation for their intended use. For example the collected HPLC fraction from the preparative HPLC, containing a compound according to the invention may be diluted with water or water containing such as, but not limited to, sodium hydroxide or hydrogen chloride. The diluted fraction as prepared is trapped on a Seppak® such as, for example but not limited to, C18, tC18, Silica or an Oasis Seppak® and the compound is preferably eluted from the Seppak® with a solvent suitable for injection in vivo, like ethanol. The obtained eluate is preferable diluted with pharmaceutically acceptable buffers such as, but not limited to 0.9% sodium chloride, sodiumdihydrogenphosphate 7.09 mM in 0.9% sodiumchloride or citrate buffer, pharmaceutically acceptable solubilisers such as, but not limited to, ethanol, tween or phospholipids and/or with pharmaceutically acceptable stabilizers or antioxidants such as, but not limited to, ascorbic acid, gentisic acid or p-aminobenzoic acid.

The invention is also directed to the following new compounds or a salt or solvate thereof.

A compound or a salt or solvate thereof according to the following formula:

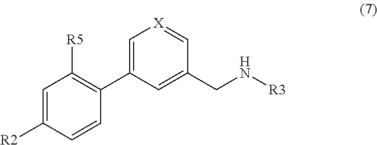

wherein X is nitrogen or carbon, R⁵ is hydrogen or an optionally fluorinated alkoxy group having 1 to 5 carbon atoms, R² is fluorine and R³ is an organic group and preferably a branched or unbranched C₁-C₆ alkyl, C₃-C₇ cycloalkyl or a methyl C₃-C₇ cycloalkyl group. Examples of alkyl groups are methyl, ethyl or n-propyl. Examples of cycloalkyl groups are, C₃-C₇ cycloalkyl and preferably cyclopentyl, cyclobutyl. Examples of methyl cycloalkyl groups are —CH₂-cyclopropyl, —CH₂-cyclobutyl, —CH₂-cyclopentyl and preferably —CH₂-cyclopropyl. The compound according to formula (7) or salt or solvate thereof may be radiolabeled. If R⁵ comprises a alkoxy group it is preferred that it comprises a ¹¹C or a ¹⁸F isotope. If R⁵ is hydrogen it is preferred that R² is a ¹⁸F isotope. Such a compound is preferably prepared according to a electrophilic substitution reaction as described below.

For electrophilic ¹⁸F-labelling reactions carrier-added elemental fluorine [¹⁸F]F₂ or acetylhypofluorite [¹⁸F]CH₃CO₂F can be used in aromatic fluorodemetallation reactions according to scheme 7a. Suitable organometallic precursors used for electrophilic substitution are aryltrimethyltin, aryltrimethylgermanium and aryltrimethylsilicon compounds as described by Coenen H H et. al., Journal of Fluorine Chemistry (1987), 36, 63-75.

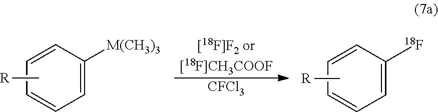

More preferably organotin derivatives are used. Starting from the above described compound a skilled person can easily prepare the compound according to formula (7), wherein R1 is hydrogen.

The invention is thus also directed to the salts and solvates of the compound according to formula (7). Suitable salts include physiologically acceptable acid addition salts such as those derived from mineral acids, but not limited to, hydrochloric, hydrobromic, phosphoric, metaphosphoric, nitric or sulphuric acids or those derived from organic acids such as, but not limited to, tartaric, fumaric, malonic, citric, benzoic, trifluoroacetic, lactic, glycolic, gluconic, methanesulphonic or p-toluenesulphonic acids.

A preferred alkoxy group for R⁵ is methoxy. Preferably R³ is —CH₂-cyclopropyl. Preferably X is nitrogen. The carbon of the methoxy group R⁵ is preferably a ¹¹C atom.

Another preferred alkoxy group R⁵ comprises an alkyl group having from one to five fluorine substituents. More preferably the alkyl group is substituted with one fluorine atom and wherein the fluorine atom is a radio labelled ¹⁸F atom.

The compounds as may be prepared from the compounds according to this invention as described above may advantageously be used as part of a pharmaceutical composition for use as a medicament and more preferably for use in the therapeutic treatment of neurodegenerative disorders, such as especially neuronal loss in hypoxia, hypoglycemia, brain or spinal cord ischemia, and brain or spinal chord trauma as well as being useful for the treatment of epilepsy, Alzheimer's disease, Amyotrophic Lateral Sclerosis, Parkinson's disease, Huntington's disease, Down's Syndrome and/or Korsakoff's disease.

The invention is also directed to compounds as may be prepared from the compounds according to this invention as described above for use as an antagonist of the NR2B binding site of the N-methyl-D-aspartate (NMDA) receptor.

The radio labelled compounds according to the invention can advantageously be used as diagnostic imaging agents for in vivo imaging of the NR2B binding site of the NMDAR complex with positron emission tomography (PET) or single photon emission computed tomography (SPECT). The NMDAR complex belongs to the ionotropic glutamate receptor family and are involved in many physiological processes. NMDAR's are heteromeric complexes which consists of four subunits namely three subtypes, NR1, in eight different splice variants, NR2, in four different subunits (NR2A-D), NR3, in two different subunits (NR3A-B) (NR1, NR2 and NR3 are typical codes used in literature for describing NMDAR's and are not to be confused with R¹, R² and R³ as used in formulas (1)-(7)). Imaging the NMDAR complex in living animal or human brain by PET or SPECT provides useful information on the role of the NMDAR complex in various neurological disorders such as, for example Alzheimer's disease, Huntington's disease, Korsakoff's disease and other neurodegenerative disorders, for example those described above.

The invention is thus also directed to a method for the in vivo diagnosis or imaging of NMDA related disease in a subject, preferably a human, comprising administration of the above described radio labelled compounds according to the invention. Administration of the compound is preferably administrated in a radiopharmaceutical formulation comprising the compound or its salt or solvate and one or more pharmaceutically acceptable excipients in a form suitable for administration to humans. The radiopharmaceutical formulation is preferably an aqueous solution additionally comprising a pharmaceutically acceptable buffer, a pharmaceutically acceptable solubiliser such as, but not limited to, ethanol, tween or phospholipids, pharmaceutically acceptable stabilizer solutions and/or antioxidants such as, but not limited to, ascorbic acid, gentisic acid or p-aminobenzoic acid.

The invention is thus also directed to a radiopharmaceutical formulation comprising the radio labelled compound according to the invention and to a radiopharmaceutical formulation comprising the radio labelled compound according to the invention for use as an in vivo diagnostic or imaging method, wherein the method is preferably positron emission tomography (PET) or single photon emission computed tomography (SPECT).

The invention shall be illustrated by means of the following non-limiting examples.

General methods (see also: D. G. Brown et al., Bioorganic Medicinal Chemistry Letters (2011), 21, 3399-3403)

Below the preparation of used examples 1-4 according to scheme 8 is described in general terms.

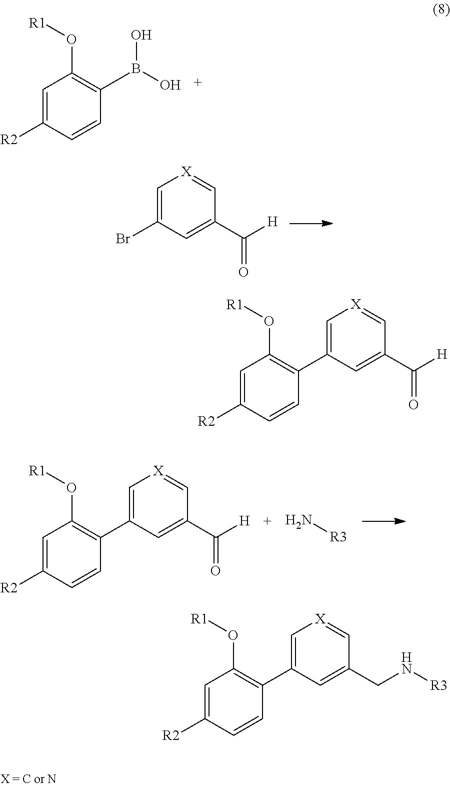

2-bromobenzaldehyde or 5-bromonicotinaldehyde (1 equivalent) was dissolved in a mixture of DME/EtOH/H₂O (7:2:1, 1M) and treated with a boronic acid (1 equivalent) followed by cesium carbonate (1.09 equivalent) and 1,1′-bis(diphenylphosphino)ferrocenedichloro palladium(II) dichloromethane complex (0.04 equivalent).

The reaction mixture was heated at 80° C. for 12 h and then partitioned between ethyl acetate and water. The organic layer was washed with saturated NaHCO₃, saturated brine and dried over MgSO₄, filtered and concentrated under reduced pressure to give a dark brown solid. The solid was purified by column chromatography (SiO₂, 0-25% ethylacetate in hexanes) to give the desired product as a white solid.

The resulting aldehyde (1 equivalent) was dissolved in dry dichloromethane along with the appropriate amine (1.5 equivalent). The solution was cooled in an ice bath, and to this was added sodium triacetoxy borohydride (1.1 to 1.5 equivalent). The reaction was stirred at room temperature for 16 h and diluted with dichloromethane. The resultant mixture was then washed with saturated NaHCO₃, saturated brine and dried over MgSO₄, filtered and concentrated under reduced pressure to give an oil. The oil was purified by columnchromatography (SiO₂, 0-25% methanol in dichloromethane) yielding the desired product as a yellow oil. The oil was dissolved in diethyl ether and a 1 M HCl in diethyl ether solution was added dropwise. The suspension was stirred for 30 minutes. The white solid was filtered and washed with diethyl ether and dried under vacuum to afford the desired product as a white solid.

### EXAMPLE 1

According to the general procedure described above N-((5-(4-fluoro-2-methoxyphenyl)pyridin-3-yl)methyl)cyclopentanamine dihydrochloride, HC-242 (see also below) was prepared starting from 5-(4-fluoro-2-methoxyphenyl)nicotinaldehyde (300 mg, 1.3 mmol).

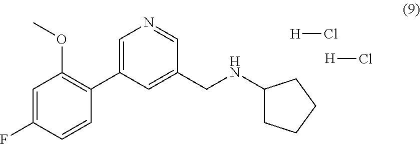

N-((5-(4-fluoro-2-methoxyphenyl)pyridin-3-yl)methyl)cyclopentanamine dihydrochloride was obtained as a white solid (196 mg, 0.53 mmol, 40%).

HR-MS: [M+H]⁺=287.1549 found; [M+H]⁺=287.1554 calc.

[¹H]-NMR (free base in CDCl₃) [250 MHz]

δ=1.285-1.459 (m, 2H, CH₂ of c-pentyl), 1.478-1.508 (m, 2H, CH₂ of c-pentyl), 1.614-1.673 (m, 2H, CH₂ of c-pentyl), 1.764-1.831 (m, 2H, CH₂ of c-pentyl), 1.993 (bs, 1H, NH), 3.027-3.131 (m, 1H, CH of c-pentyl), 3.723 (s, 3H, CH₃), 3.762 (s, 2H, CH₂—N), 6.627-6.710 (m, 2H, 2×φ-H), 7.161-7.260 (s, 1H, φ-H), 7.720 (s, 1H, φ-H), 8.429 (s, 1H, φ-H), 8.532 (s, 1H, φ-H).

### EXAMPLE 2

According to the general procedure described above 1-cyclopropyl-N-((5-(4-fluoro-2-methoxyphenyl)pyridin-3-yl)methyl)methanamine dihydrochloride was prepared starting from 5-(4-fluoro-2-methoxyphenyl)nicotinaldehyde (300 mg, 1.3 mmol).

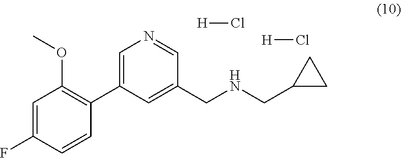

1-cyclopropyl-N-((5-(4-fluoro-2-methoxyphenyl)pyridin-3-yl)methyl)methanamine dihydrochloride was obtained as a white solid (185 mg, 0.52 mmol, 40%).

HR-MS: [M+H]⁺=287.1550 found; [M+H]⁺=287.1554 calc.

[¹H]-NMR (free base in CDCl₃) [250 MHz]

δ=0.6045-0.6643 (m, 2H, CH₂ of c-propyl), 0.9705-1.0429 (m, 2H, CH₂ of c-propyl), 1.4241-1.7717 (m, 1H, CH of c-propyl), 2.4268 (bs, 1H, NH), 3.0280-3.0553 (m, 2H, N—CH₂-c-propyl), 4.3177 (s, 3H, CH₃—O), 4.3952 (s, 2H, CH₂—N), 7.2107-7.3009 (m, 2H, 2×φ-H), 7.7472-7.8076 (m, 1H, φ-H), 8.2944-8.3101 (m, 1H, φ-H), 9.0394 (s, 1H, φ-H), 9.1145 (s, 1H, φ-H).

### EXAMPLE 3

According to the general procedure described above N-((4′-fluoro-2′-methoxy-[1,1′-biphenyl]-3-yl)methyl)cyclopentanamine hydrochloride

(see also below) was prepared starting from 4′-fluoro-2′-methoxy-[1,1′-biphenyl]-3-carbaldehyde (300 mg, 1.3 mmol).

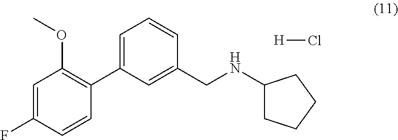

N-((4′-fluoro-2′-methoxy-[1,1′-biphenyl]-3-yl)methyl)cyclopentanamine hydrochloride was obtained as a white solid (203 mg, 0.60 mmol, 46%).

[¹H]-NMR (HCl salt in DMSO-d6) [500 MHz]

δ=1.514-1.525 (m, 2H, CH₂ of c-pentyl), 1.690-1.746 (m, 4H, 2×CH₂ of c-pentyl), 1.969-1.983 (m, 2H, CH₂ of c-pentyl), 3.426-3.467 (m, 1H, CH of c-pentyl), 3.786 (s, 3H, CH₃), 4.141 (s, 2H, CH₂—N), 6.866-6.905 (dt, 1H, φ-H, 42=8.38 Hz; 43=2.47 Hz), 7.034-7.061 (dd, 1H, φ-H, 43=2.47 Hz), 7.161-7.260 (dt, 1H, φ-H, 42=8.38 Hz), 7.431-7.538 (m, 3H, 3×φ-H), 7.647 (s, 1H, φ-H), 9.321 (s, 2H, NH₂⁺).

[¹³C]-NMR (HCl salt in DMSO-d6) [500 MHz]

δ=24.25 (2×CH₂ of c-pentyl), 29.67 (2×CH₂ of c-pentyl), 49.95 (CH₂—N), 56.61 (CH₃), 58.55 (CH of c-pentyl), 100.60 (CH-φ), 107.56 (CH-φ), 126.10 (C—F)+132.68 (C—F), 128.98 (CH-φ), 130.33 (CH-φ), 131.50 (CH-φ), 132.02 (CH-φ), 138.16 (C-φ), 158.00 (C-φ), 162.26 (C-φ), 164.20 (C-φ).

### EXAMPLE 4

According to the general procedure described above 1-cyclopropyl-N-((4′-fluoro-2′-methoxy-[1,1′-biphenyl]-3-yl)methyl)methanamine hydrochloride (see also below) was prepared starting from 4′-fluoro-2′-methoxy-[1,1′-biphenyl]-3-carbaldehyde (300 mg, 1.3 mmol).

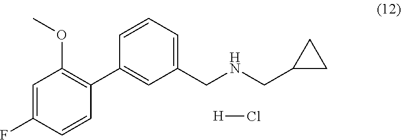

1-cyclopropyl-N-((4′-fluoro-2′-methoxy-[1,1′-biphenyl]-3-yl)methyl)methanamine hydrochloride was obtained as a white solid (170 mg, 0.53 mmol, 41%).

[1H]-NMR (HCl salt in DMSO-d6) [500 MHz]

δ=0.367-0.385 (m, 2H, CH₂ of c-propyl), 0.551-0.588 (m, 2H, CH₂ of c-propyl), 1.101-1.160 (m, 1H, CH of c-propyl), 2.804-2.818 (d, 2H, N—CH₂-c-propyl, 42=7.33 Hz), 3.788 (s, 3H, CH₃), 4.160 (s, 2H, CH₂—N), 6.864-6.9035 (dt, 1H, φ-H, 42=8.37 Hz; 43=2.46 Hz), 7.033-7.061 (dd, 1H, φ-H, 43=2.46 Hz), 7.349-7.380 (dt, 1H, φ-H, J_{1,2}=8.37 Hz), 7.429-7.525 (m, 3H, 3×φ-H), 7.640 (s, 1H, φ-H), 9.407 (s, 2H, NH₂⁺).

### General Methods

Below the demethylation of examples 1-4 according to scheme 13 are described in general terms

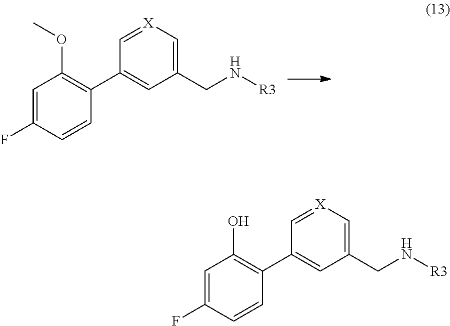

To a solution of a 5-(4-fluoro-2-methoxyphenyl)derivative in dry dichloromethane (Ratio: 5 ml/mmol) was added boron tribromide in dichloromethane (1M) (5 equivalents) dropwise at −78° C. After addition the reaction mixture was stirred for 16 h at RT. Then MeOH was added at 0° C. and the reaction was stirred for 5 min. Subsequently, water and ethyl acetate were added. The mixture was fractionated and the aqueous layer was extracted with ethyl acetate. The combined extracts were dried with MgSO₄ and evaporated to dryness. Dichloromethane is added and the suspension is stirred for 5 min, filtered and washed with dichloromethane and dried under vacuum at 50° C. to afford the desired product as a white solid.

### EXAMPLE 5

According to the general procedure described above 2-(5-((cyclopentylamino)methyl)pyridin-3-yl)-5-fluorophenol (see also below) was prepared starting from N-((5-(4-fluoro-2-methoxyphenyl)pyridin-3-yl)methyl)cyclopentanamine (420 mg, 1.40 mmol).

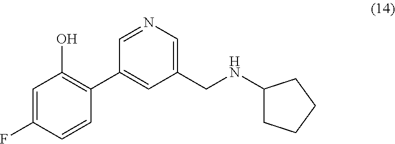 [SMILES: Oc1cc(F)ccc1-c1cncc(CNC2CCCC2)c1]

2-(5-((cyclopentylamino)methyl)pyridin-3-yl)-5-fluorophenol was obtained as a white solid (298 mg, 1.04 mmol, 74%).

TLC: dichloromethane/methanol 9/1 v/v R_{f}=0.53 [ninhydrine positive].

HR-MS: [M+H]⁺=287.1549 found; [M+H]⁺=287.1554 calc.

[¹H]-NMR in DMSO-d6 [250 MHz]

δ=1.560-1.753 (m, 6H, 3×CH₂ of c-pentyl), 2.025-2.078 (m, 2H, CH₂ of c-pentyl), 3.679-3.682 (m, 1H, CH of c-pentyl), 4.300-4.574 (m, 2H, CH₂—N), 6.808-6.988 (m, 2H, 2×φ-H), 7.442-7.726 (m, H, φ-H), 8.391-9.087 (m, 4H, 1×OH+3×φ-H), 9.570-9.876 (m, H, φ-H), 10.574-11.172 (m, 1H).

HPLC analysis: GraceSmart RP 18; 5 μm; 4.6×250 mm; Flow=1 mL/min; UV=254 nM.

Eluent=H₂O/acetonitrile (85/15)+0.1% TFA. Retention time=6.52 min.

HR-MS: [M+H]⁺=287.1549 found; [M+H]⁺=287.1554 calc.

### General Methods

Below the fluoroalkylation of phenol derivatives according to scheme 15 is described in general terms.

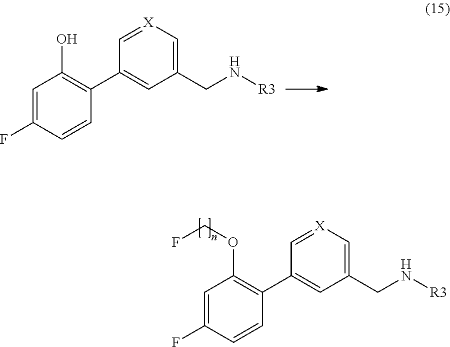

To a solution of the appropriate phenol in DMSO (Volume: 7 mL/mmol) were added potassium carbonate (2 equivalents), potassium iodide (0.1 equivalents), and 1-bromo-2-fluoroalkane (1.1 equivalents). The reaction mixture was stirred at 85° C. for 1.5 h. The reaction was diluted with water and extracted with ethyl acetate (3 times). The combined organic layers were washed with brine, dried with magnesium sulfate and evaporated to dryness to obtain a brown oil. The brown oil was purified by column chromatography (SiO₂, 0-10% methanol in dichloromethane) yielding the desired product as a yellow oil.

The oil was dissolved in diethyl ether and a 1 M HCl in diethyl ether solution was added dropwise. The suspension was stirred for 30 minutes. The white solid was filtered and washed with diethyl ether and dried under vacuum to afford the desired product as a white solid.

### EXAMPLE 6

According to the general procedure described above N-((5-(4-fluoro-2-(fluoromethoxy)phenyl)pyridin-3-yl)methyl)cyclopentanamine dihydrochloride (see also below) was prepared starting from 2-(5-((cyclopentylamino)methyl)pyridin-3-yl)-5-fluorophenol (75 mg, 0.26 mmol).

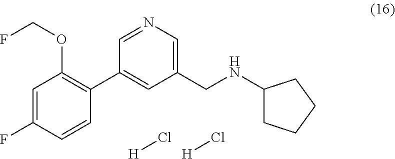

N-((5-(4-fluoro-2-(fluoromethoxy)phenyl)pyridin-3-yl)methyl)cyclopentanamine dihydrochloride was obtained as a white solid (21.3 mg, 0.054 mmol, 21%).

[1H]-NMR (free base in CDCl3) [500 MHz]

δ=1.355-1.392 (m, 2H, CH₂ of c-pentyl), 1.526-1.557 (m, 2H, CH₂ of c-pentyl), 1.686-1.717 (m, 2H, CH₂ of c-pentyl), 1.829-1.879 (m, 2H, CH₂ of c-pentyl), 3.136 (pentet, 1H, CH of c-pentyl, J=6.63 Hz), 3.834 (s, 2H, CH₂), 5.651 (d, 2H, CH₂—F, J_{H—F}=53.9 Hz), 6.899-6.937 (m, 1H, φ-H), 6.984-7.009 (m, 1H, φ-H), 7.301-7.331 (m, 1H, φ-H), 7.759 (s, 1H, pyridyl-H), 8.519 (s, 1H, pyridyl-H), 8.569 (s, 1H, pyridyl-H).

### EXAMPLE 7

According to the general procedure described above N-((5-(4-fluoro-2-(2-fluoroethoxyl)phenyl)pyridin-3-yl)methyl)cyclopentanamine dihydrochloride (see also below) was prepared starting from 2-(5-((cyclopentylamino)methyl)pyridin-3-yl)-5-fluorophenol (83 mg, 0.29 mmol).

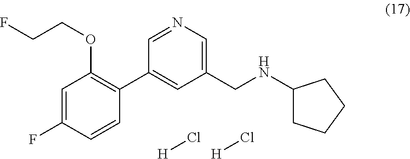

N-((5-(4-fluoro-2-(2-fluoroethoxyl)phenyl)pyridin-3-yl)methyl)cyclopentanamine dihydrochloride was obtained as a white solid (38.7 mg, 0.095 mmol, 33%).

[1H]-NMR (free base in CDCl3) [250 MHz]

δ=1.279-1.855 (m, 8H, 4×CH₂ of c-pentyl), 3.075 (pentet, 1H, CH of c-pentyl, J=6.55 Hz), 3.754 (s, 2H, CH₂), 4.068-4.211 (dd, 2H, CH₂—C—F, J_{H—F}=27.66 Hz), 4.508-4.730 (dd, 2H, CH₂—F, J_{H—F}=47.29 Hz), 6.620-6.766 (m, 2H, 2×φ-H), 7.217-7.277 (m, 1H, φ-H), 7.792 (s, 1H, pyridyl-H), 8.426 (s, 1H, pyridyl-H), 8.534 (s, 1H, pyridyl-H).

### General Methods

Below the [¹¹C]methylation of phenol derivatives according to scheme 18 is described in general terms.

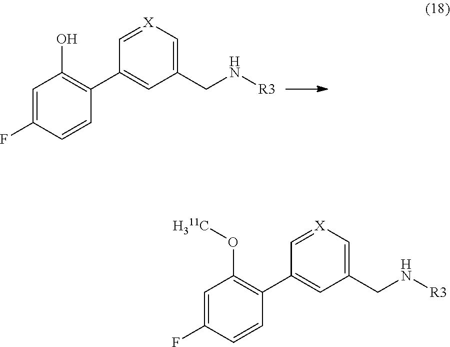

The appropriate phenol (1 mg) was reacted with [¹¹C]CH₃I (prepared under the general conditions known by a person skilled in the art) in dimethylsulfoxide (300 μL) in the presence of potassium carbonate (10 mg, 72 μmol) for 5 minutes at 80° C. After reaction the mixture is quenched with 50% Acetonitrile in water (300 μL) before purification by preparative HPLC. The fraction containing product was collected and diluted with water (50 mL). The solution was concentrated on a tC18plus Seppak, rinsed with water (20 mL), subsequently eluted with ethanol (96%, 1 mL) and diluted with a solution of 7.11 mM NaH₂PO₄ in 0.9% NaCl (w/v in water), pH 5.2 (9 mL) to give a final solution of 9.6% ethanol.

### EXAMPLE 8

According to the general procedure described above N-((5-(4-fluoro-2-¹¹C-methoxyphenyl)pyridin-3-yl)methyl)cyclopentanamine, [¹¹C]HC-242 (see also below) was prepared in high radiochemical yield and radiochemical purity (>99%).

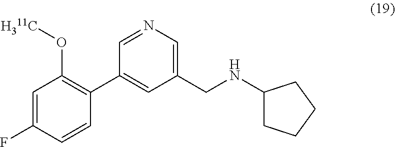 [SMILES: [11CH3]Oc1cc(F)ccc1-c1cncc(CNC2CCCC2)c1]

HPLC analysis: GraceSmart RP 18; 5 μm; 4.6×250 mm; Flow=1 mL/min; UV=254 nM. Eluent=H₂O/acetonitrile (85/15)+0.1% TFA. Retention time=4.10 min.

### General Methods

Below the [¹⁸F]fluoroalkylation of phenol derivatives according to scheme 20 is described in general terms.

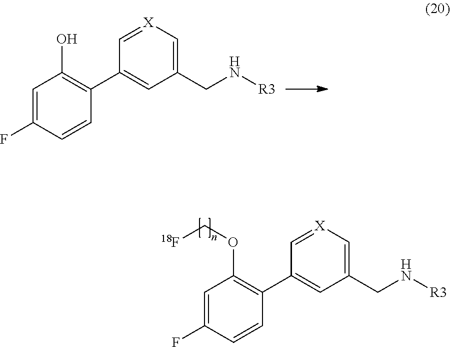

The appropriate phenol (1 mg) was reacted with [¹⁸F]F-alkyl-Br or with [¹⁸F]F-alkyl-OTf (prepared under the general conditions known by a person skilled in the art) in dimethylformamide (250 μL) in the presence or absence of potassium iodate (0.5 mg) and sodium hydride (1 mg) for 15 minutes at 100° C. After reaction the mixture is quenched with 50% Acetonitrile in water (300 μL) before purification by preparative HPLC. The fraction containing product was collected and diluted with water (50 mL). The solution was concentrated on a tC18plus Seppak, rinsed with water (20 mL), subsequently eluted with ethanol (96%, 1 mL) and diluted with a solution of 7.11 mM NaH₂PO₄ in 0.9% NaCl (w/v in water), pH 5.2 (9 mL).

### EXAMPLE 9

According to the general procedure described above N-((5-(4-fluoro-2-(2-[¹⁸F]fluoromethoxy)phenyl)pyridin-3-yl)methyl)cyclopentanamine (see also below) was prepared from [¹⁸F]CH2FBr in high radiochemical yield and radiochemical purity (>99%).

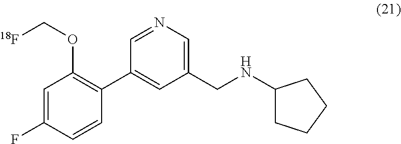 [SMILES: Fc1ccc(-c2cncc(CNC3CCCC3)c2)c(OC[18F])c1]

### EXAMPLE 10

According to the general procedure described above N-((5-(4-fluorophenyl)pyridin-3-yl)methyl)cyclopentanamine dihydrochloride was prepared starting from 5-(4-fluorophenyl)nicotinaldehyde (500 mg, 2.48 mmol).

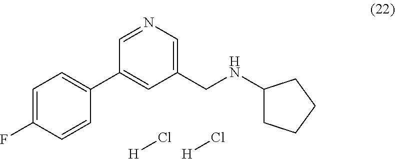

N-((5-(4-fluorophenyl)pyridin-3-yl)methyl)cyclopentanamine dihydrochloride was obtained as a white solid (507 mg, 1.48 mmol, 59%).

[¹H]-NMR (free base in CDCl₃) [250 MHz]

δ=1.345-1.937 (m, 8H, 4×CH₂ of c-pentyl), 3.146 (pentet, 1H, CH of c-pentyl, J=6.56 Hz), 3.849 (s, 2H, CH₂), 7.114-7.260 (m, 2H, 2×φ-H), 7.516-7.596 (m, 2H, 2×φ-H), 7.834 (s, 1H, pyridyl-H), 8.527 (s, 1H, pyridyl-H), 8.682 (s, 1H, pyridyl-H).

### EXAMPLE 11

According to the general procedure described at formula (7a) above N-((4′-fluoro-[1,1′-biphenyl]-3-yl)methyl)cyclopentanamine hydrochloride was prepared starting from 4′-fluoro-[1,1′-biphenyl]-3-carbaldehyde (450 mg, 2.25 mmol).

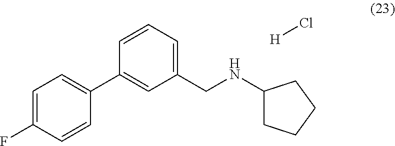

N-((4′-fluoro-[1,1′-biphenyl]-3-yl)methyl)cyclopentanamine hydrochloride was obtained as a white solid (156 mg, 0.51 mmol, 23%).

HR-MS: [M+H]⁺=270.1649 found; [M+H]⁺=270.1653 calculated

[¹H]-NMR(HCl salt in DMSO-d6) [250 MHz].

δ=1.506-1.531 (m, 2H, CH₂ of c-pentyl), 1.753-1.811 (m, 4H, 2×CH₂ of c-pentyl), 1.944-2.004 (m, 1H, CH₂ of c-pentyl), 3.423-3.478 (m, 1H, CH of c-pentyl), 4.170 (s, 2H, CH₂—N), 7.287-7.358 (m, 2H, 2×φ-H), 7.467-7.580 (m, 2H, 2×φ-H), 7.673-7.703 (m, 1H, φ-H), 7.757-7.814 (m, 2H, 2×φ-H), 7.995 (s, 1H, φ-H), 9.487 (s, 1.9H, NH₂⁺).

### EXAMPLE 12

According to the general procedure described at formula (7a) above 1-cyclopropyl-N-((5-(4-fluorophenyl)pyridin-3-yl)methyl)methanamine dihydrochloride was prepared starting from 5-(4-fluorophenyl)nicotinaldehyde (500 mg, 2.49 mmol).

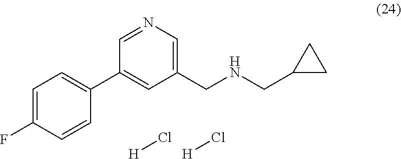

N-((4′-fluoro-[1,1′-biphenyl]-3-yl)methyl)cyclopentanamine hydrochloride was obtained as a white solid (506 mg, 1.54 mmol, 62%).

HR-MS: [M+H]+=257.1641 found; [M+H]+=257.1454 calculated

[¹H]-NMR (free base in CDCl3) [500 MHz]

δ=0.110-0.140 (m, 2H, CH2 of c-propyl), 0.483-0.520 (m, 2H, CH2 of c-propyl), 0.962-1.022 (m, 1H, CH of c-propyl), 2.524-2.538 (d, 2H, N—CH2-c-propyl), 3.897 (s, 2H, CH2-N), 7.138-7.185 (m, 2H, 2×φ-H), 7.539-7.579 (m, 2H, 2×φ-H), 7.852 (t, 1H, pyridyl-H), 8.528 (d, 1H, pyridyl-H), 8.687 (d, 1H, pyridyl-H).

[¹³C]-NMR (free base in CDCl3) [500 MHz]

δ=3.57 (2×CH2 of c-propyl), 11.33 (CH of c-propyl), 51.18 (CH2-c-propyl), 54.71 (CH2-N), 116.07 (CH-φ), 116.25 (CH-φ), 128.98 (CH-φ), 129.04 (CH-φ), 134.06 (C-φ), 134.22 (C-pyridyl), 135.64 (C-φ), 135.88 (C-φ), 146.95 (C-pyridyl), 148.52 (C-pyridyl), 163.04 (C-φ).

### EXAMPLE 13

### Membrane Preparation

Male Wistar rats (180-200 g) were killed by decapitation. The forebrains were rapidly removed and homogenized using a DUALL tissue homogenizer (10 strokes, 2000 rpm), in a 10-fold excess (v/w) of ice-cold 0.25 M sucrose. The nuclei and cell debris were removed by centrifugation (10 min×400×g), in a Beckman refrigerated ultracentrifuge (rotor 60Ti). The resulting pellet was rehomogenized in 5 vol 0.25 sucrose and recentrifuged. The combined supernatants were diluted in Tris-acetate buffer (50 mM, pH 7.4) to a final dilution of 40 v/w, and centrifuged for 30 min×200,000×g, in order to obtain membranes from the cell surface, mitochondrial, and microsomal fractions. The pellet was resuspended in 20 volumes of Tris-HCl+0.01% Triton buffer (pH 7.4), kept at 37° C. for 10 min, and recentrifuged. The final pellet was suspended in Tris-HCl buffer (dilution 5, pH 7.4), and stored at −80° C. in 5 ml aliquots. On the day of each experiment, membranes were thawed to room temperature and washed twice by centrifugation (30 min×200,000×g). After the final centrifugation, pellets were suspended in 80 volumes (v/w) of 50 mM Tris-HCl buffer (pH 7.4).

### EXAMPLE 14

### Competition Binding Assays

The affinity of novel compounds for the ifenprodil binding site of NR2B subunit containing NMDA receptors was determined by measuring the ability of various concentrations of unlabelled ligand to inhibit the specific binding of 5 nM [³H]ifenprodil (specific activity: 40 Ci/mmol). Unlabelled ligands were dissolved as 10 mM stock solutions in DMSO, and used in a concentration range from 10⁻⁴ to 10⁻¹²M. Competition binding experiments were carried out on ice, in a final volume of 500 μl assay buffer (50 mM Tris-HCl, pH 7.4), containing 5 μM GBR 12909 to block a receptor binding. The incubation mixture was composed of 400 μl of the membrane suspension, containing a total amount of 2.5 mg original wet weight of tissue, 50 μl of [³H]ifenprodil, and 5 μl of unlabelled drug solution (final DMSO concentration: 1%). Nonspecific binding was determined in the presence of 10 μM Ro 25-6981. Incubations were terminated after 2 hr by filtration through 0.3% PEI-embedded Whatman GF/B filters, using a 48-well Brandel harvester. The filters were washed three times with 3 ml of ice cold Tris-HCl buffer (pH 7.4), and radioactivity was determined by liquid scintillation spectrometry in 5 ml of Optiphase-HiSafe 3, at an efficiency of 40%.

### EXAMPLE 15

### Data Analysis

Kᵢ values were determined by nonlinear regression analysis using the equation: log EC50=log [10̂log Ki*(1+RadioligandNM/HotKdNM)], (GraphPad Software Inc., San Diego, Calif.).

Table 1 below shows the affinity of compounds for the NMDAR NR2B binding site against [³H]ifenprodil. In this table the Ki for the different compounds is stated. The Ki as measured against 5 nM [³H]ifenprodil and represent the mean±SEM Ki values of 2-6 independent determinations, each conducted in triplicate.

TABLE 1
Affinity data for the NR2B binding site on the NMDAR

| Compound | Structure | Kᵢ in nM ^{x} |
|---|---|---|
| Example 1 | 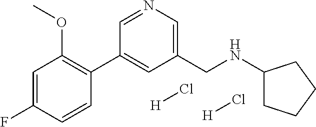 | 12.4 ± 2.1 (12 nM ^{#}) |
| Example 2 | 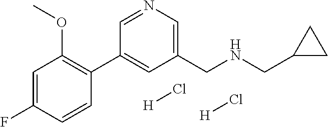 | 8.7 ± 1.2 |
| Example 3 | 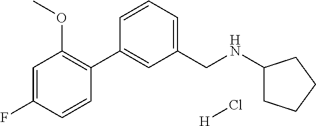 | 60.0 ± 2.9 (48 nM ^{#}) |
| Example 4 | 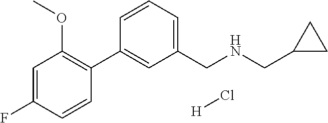 | 67.7 ± 8.1 |
| Example 5 | 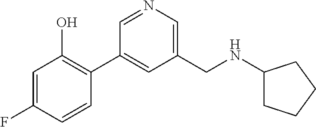 [SMILES: Oc1cc(F)ccc1-c1cncc(CNC2CCCC2)c1] | 1300 ± 600 |
| Example 6 | 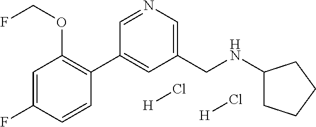 | 15.4 ± 1.3 |
| Example 7 | 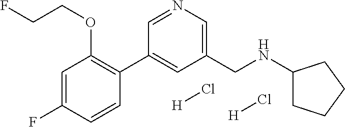 | 156.5 ± 21.8 |
| Example 10 | 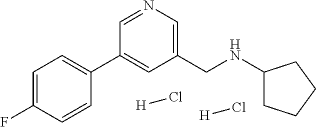 | 3.3 ± 1.9 |
| Example 11 | 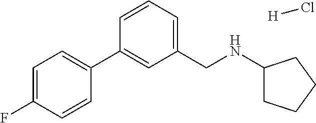 [SMILES: Fc1ccc(-c2cccc(CNC3CCCC3)c2)cc1 ; Cl] | 13.1 ± 1.8 |
| Example 12 | 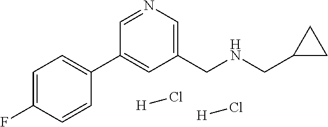 | 2.9 ± 0.3 |

^{x} displacement of tritium labelled ifenprodil
^{#} Values listed in D.G. Brown et al., Bioorganic Medicinal Chemistry Letters (2011), 21 3399-3403)

### EXAMPLE 16

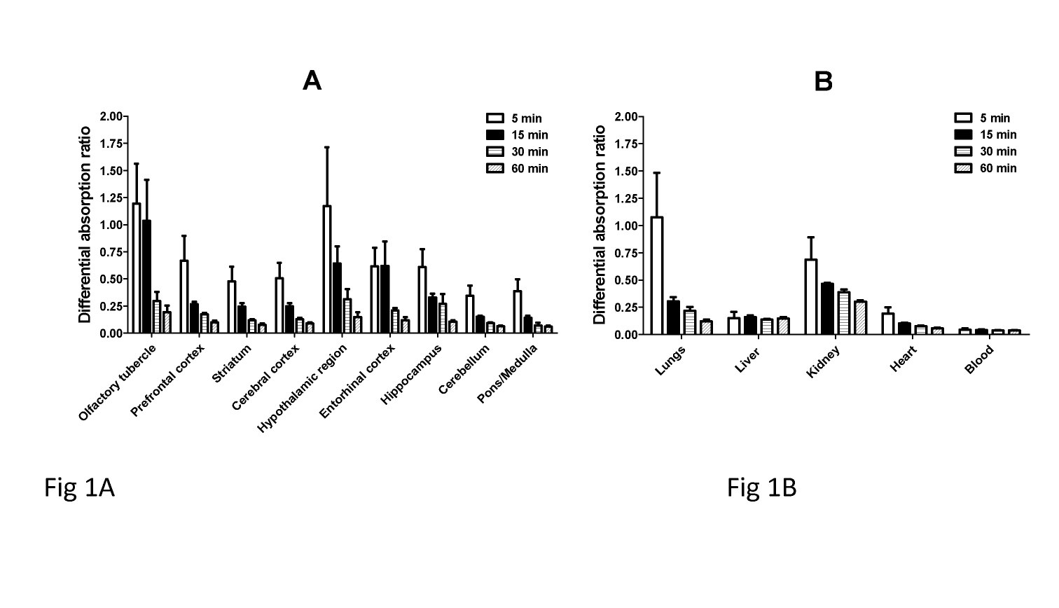

Biodistribution of [¹¹C]HC-242 as prepared in Example 8 in anaesthetized mice Male, 7-9 weeks old B6C3F1/J mice were used for biodistribution experiments. Animals were anaesthetized with a 1:2:1 mixture of ffm [fentanyl/fluanisone (Hypnorm™; Vetapharma, Leeds, UK), sterile water, midazolam (Dormicum™; Actavis, Harnarfjordur, Iceland)]. The anaesthetic was administered intraperitoneally (11 ml/kg), 5 min prior to radiotracer administration. [¹¹C]HC-242 (60.7±3.4 MBq at t₀) was injected via the tail vein, in a saline solution containing 10% ethanol (5 ml/kg). Following the injections, mice were killed by cervical dislocation at 5, 15, 30, or 60 min (n=4/time point). At each time point, blood was obtained by heart punctures and selected organs, including the heart, liver, kidneys, lungs and brain were removed. The brain was further dissected into frontal cortex, olfactory tubercle, striatum, cerebral cortex (bregma 1.70-0.02 mm), entorhinal cortex, hippocampus, hypothalamus, cerebellum, and pons/medulla oblongata. All organs and brain areas were weighed, and recovered radioactivity was determined in a 1282 Compugamma CS (LKB Wallac, Turku, Finland), using 5×10 μl aliquots of the injected formulation as standard. Results are expressed as the differential absorption ratio (DAR): (cpm recovered/g tissue)/(cpm injected/g body weight). Two-way repeated measures ANOVA, followed by Fisher's LSD post-hoc comparisons, was used for analysis of radiotracer uptake in different brain regions (or organs), at different time points.

[¹¹C]HC-242 biodistribution results in anaesthetized mice are presented in FIG. 1. FIG. 1 shows the biodistribution of [¹¹C]HC-242 in the CNS (A) and in selected organs (B).

The brain uptake of [¹¹C]HC-242 was higher both at 5 and 15 min following injection, compared with all other time points (FIG. 1A; P<0.001, LSD post hoc tests). Significantly higher levels of uptake were observed in the olfactory tubercle and the hypothalamus, compared with all other brain regions at 5 min, and with the cerebellum and pons/medulla oblongata at 15 min post-injection (LSD tests). Radioactivity cleared rapidly from the brain, and no region-significant differences in [¹¹C]HC-242 accumulation were observed 30 or 60 min following tracer injection (P>0.05, LSD tests). The ratio of mean radioligand uptake in forebrain regions to the cerebellum was maintained above 1.9 throughout the study, with the highest ratio of 3.2±0.7 measured at 15 min post-injection. DAR values at 15 min were 1.03±0.37 for the olfactory tubercle, 0.64±0.16 for the hypothalamus, 0.62±0.22 for the entorhinal cortex, 0.33±0.03 for the hippocampus, 0.26±0.02 for the frontal cortex, 0.25±0.03 for the cerebral cortex, 0.24±0.03 for the striatum, 0.14±0.02 for the pons/medulla oblongata, and 0.14±0.01 for the cerebellum. Repeated measures ANOVA confirmed significant main effects of brain region [F_{(8,81)}=2.9, P<0.01] and time [F_{(3,81)}=23.7, P<0.001] on the uptake of [¹¹C]HC-242, with nearly significant time x region interaction effects [F_{(24,81)}=2.3, P=0.06].

The organ uptake of [¹¹C]HC-242 was higher 5 min post-injection compared with all other time points (P<0.001), and was increased in the lungs and the kidneys compared with all other organs examined (P<0.001, LSD posttests; FIG. 1B). The highest accumulation of radioactivity was measured in the lungs (1.07±0.33) and the kidneys (0.68±0.17), 5 min following [¹¹C]HC-242 injection. DAR values at 5 min were 0.04±0.01, 0.15±0.05 and 0.19±0.04 for the blood, liver and heart, respectively (P>0.05 compared with all other time points, LSD posttests). Activity was rapidly cleared from the periphery, and no significant differences in [¹¹C]HC-242 uptake were observed between organs at 30 or 60 min post-injection (P>0.05; LSD posttests). ANOVA confirmed significant main effects of organ [F_{(4,45)}=13.7, P<0.001] and time [F_{(3,45)}=7.9, P<0.001], as well as significant organ x time interaction effects [F_{(12,45)}=3.0, P<0.01] on the uptake of [¹¹C]HC-242.

### EXAMPLE 17

### In Vitro Autoradiography

Brain sections of drug-naïve, 7-9 weeks old, male B6C3F1/J mice were used for autoradiography of [¹¹C]HC-242 as prepared in Example 8 binding sites (n=3). 20 μm thick coronal brain sections were thawed from −20° C. to room temperature, pre-washed 2×10 min in assay buffer (50 mM Tris-HCl, pH 7.4), and dried. The sections were then incubated at room temperature for 30 min, in assay buffer containing [¹¹C]HC-242 (>30 nM; specific activity >30 GBq/μmol at t₀). To determine non-specific binding, a series of immediately adjacent brain sections was incubated in the same buffer, in the presence of 1 μM Ro25-6981. Incubations were terminated by 1 min washing into ice-cold assay buffer (pH 7.4), followed by a rapid rinse in ice-cold demi water. The sections were then air dried and opposed overnight to Kodak BioMax MR-1 film, which was developed in Kodak D19 developer and fixed with Kodak rapid fixer (Sigma-Aldrich, Zwijndrecht, the Netherlands). Analysis of [¹¹C]HC-242 binding was performed by video-based computerised densitometry, using an MCID image analyser (InterFocus Imaging, Linton, UK). Specific binding was quantified following the subtraction of total from non-specific binding images. Data are presented as the mean±SEM relevant optical density (ROD) values of specific [¹¹C]HC-242 binding.

### [¹¹C]HC-242 In Vitro Autoradiography Results

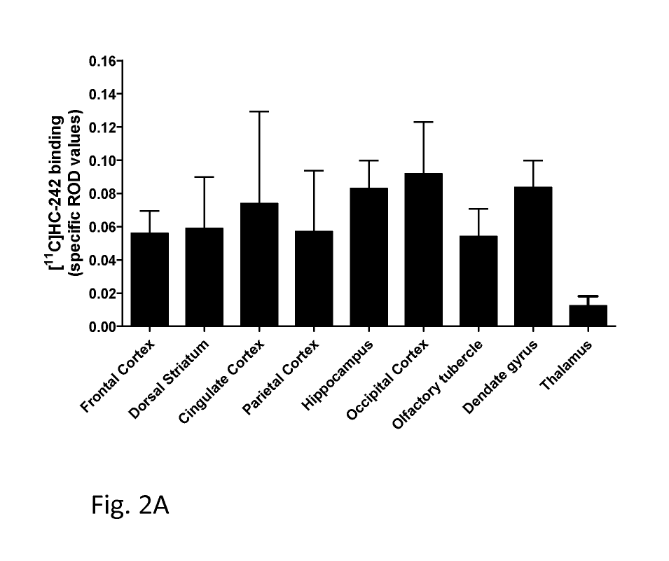

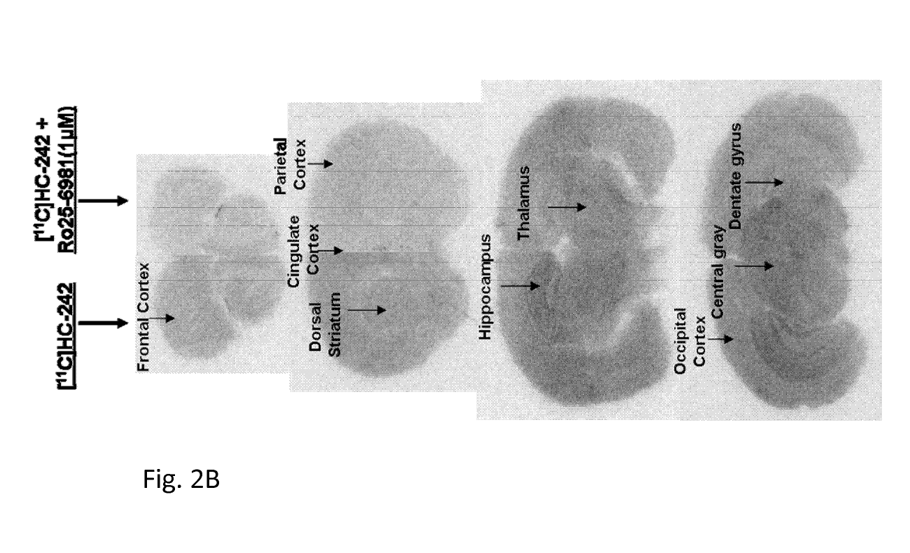

Brain sections of drug-naive mice were labeled with [¹¹C]HC-242, alone or in the presence of 1 μM Ro25-6981. The distribution of [¹¹C]HC-242 binding sites is shown in FIGS. 2 (A&B). FIG. 2A shows the quantitative in vitro autoradiography of [¹¹C]HC-242. FIG. 2B shows representative images.

Specific labelling was high in areas of the cerebral cortex, hippocampus, olfactory tubercle, and striatum, low in the thalamus, and absent in subthalamic brain regions. The mean percentage of specific binding across all brain regions analyzed was 24.0±3.5%, and ranged from 39.6±10.4% in the olfactory tubercle to 3.3±2.1% in the thalamus. High levels of non-specific labelling were observed in the thalamus and the central gray.

### EXAMPLE 18

### Blocking of [¹¹C]HC-242 in Anaesthetized Mice

To visualize the distribution and selectivity of [¹¹C]HC-242 as prepared in Example 8 for the NR2B site of NMDA receptors, B6C3F1/J mice were anaesthetized with ffm, as described in Example 17. Pairs of mice were injected with either saline or Ro25-6981 (10.0 mg/kg, i.p.), 20 min before a tail-vein injection of [¹¹C]HC-242 (n=4 pairs; 70.1±14.6 MBq at t₀). 15 min after radiotracer injection, mice were killed by cervical dislocation, and the brains were removed, frozen in liquid nitrogen and processed for quantitative autoradiography. 20 μm coronal brain sections from control and Ro25-6981 treated mice were obtained at −21° C. in a Leica CM3050S cryostat (Leica Microsystems, Rijswijk, the Netherlands), and were immediately opposed to phosphor storage screens. The screens were read in a Storm™ 860 Phosphorimager on the following day, and the images obtained were analyzed using ImageQuant TL 8.1 software (GE Healthcare Europe GmbH, Diegem, Belgium). A series of immediately adjacent brain sections were opposed to Kodak Biomax MR-1 film, which was developed after overnight exposure, to obtain high resolution images. Specific uptake was defined as the difference in brain accumulation of [¹¹C]HC-242 between control and Ro25-6981 treated animals. Analysis of radiotracer uptake in control and blocked mice across different brain regions was performed using two-way ANOVA.

### Blocking Results of [¹¹C]HC-242 in Anaesthetized Mice

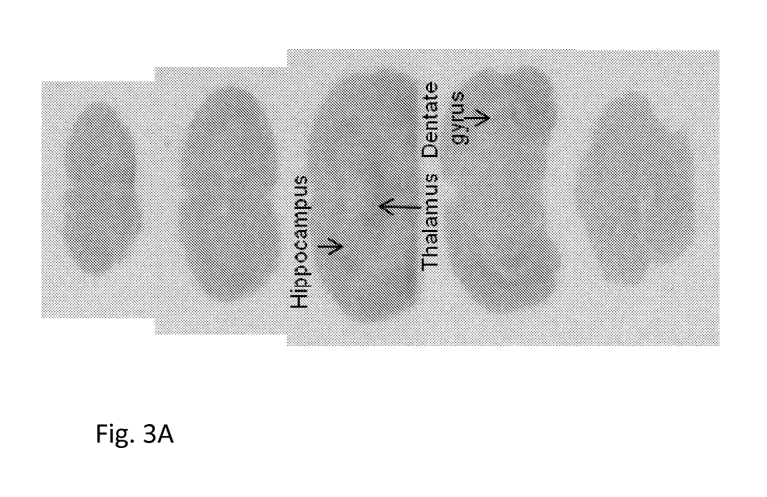

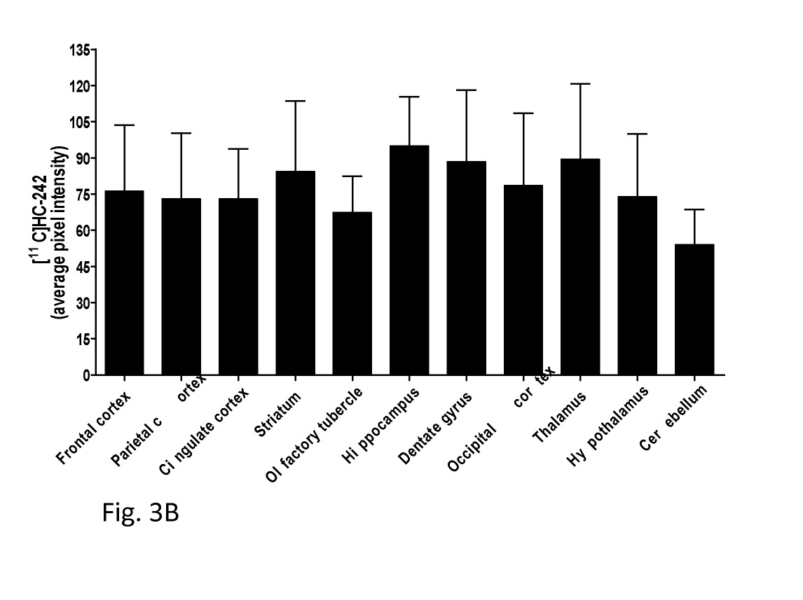

FIG. 3 shows the localization (A) and quantification (B) of [¹¹C]HC-242 uptake in brain sections of control mice. Clear labelling of the thalamus and of the hippocampal formation was observed. The highest ratios of region-to-cerebellum uptake were measured in the hippocampus (1.76), thalamus (1.66), and the dentate gyrus (1.64) of control mice. Pretreatment with Ro25-6981 had inconsistent effects on the brain uptake of [¹¹C]HC-242. In pairs of mice that were administered with identical amounts of radiolabeled compound, Ro25-6981 induced either a mean 22.3±1.9% decrease or 29.4±4.2% increase in radioactivity uptake, across all brain regions analyzed. Two-way ANOVA showed no significant main effect of antagonist administration on [¹¹C]HC-242 accumulation (treatment: [F_{(1,61)}=0.2, P>0.05]). Two-tailed t-tests, conducted on individual data sets, confirmed the variable effects of Ro25-6981 administration on the brain uptake of [¹¹C]HC-242. Table 2 summarizes the conditions and results of 4 independent studies.

TABLE 2
Pretreatment with Ro25-6981 did not consistently
block the brain uptake of [¹¹C]HC-242

| | | Activity | Specific | | | |
|---|---|---|---|---|---|---|
| Experi- | | injected | activity | [¹¹C]HC- | | |
| ment | Condi- | at t₀ | at t₀ | 242 (μM | mean % | P value |
| No | tion | (MBq) | (MBq/nmol) | at t₀) | change | (t-tests) |
| 1 | Un- | 32.23 | 5.16 | 23.7 | −22.5 | <0.001 |
| | blocked | | | | | |
| | Blocked | 30.65 | 5.02 | | | |
| 2 | Un- | 36.72 | 47.75 | 2.6 | −21.9 | <0.01 |
| | blocked | | | | | |
| | Blocked | 35.59 | 46.28 | | | |
| 3 | Un- | 89.09 | 22.82 | 14.6 | 33.3 | <0.05 |
| | blocked | | | | | |
| | Blocked | 87.66 | 22.28 | | | |
| 4 | Un- | 124.15 | 76.24 | 5.8 | 25.8 | <0.01 |
| | blocked | | | | | |
| | Blocked | 125.05 | 79.11 | | | |

### EXAMPLE 19

### Blocking of [¹¹C]HC-242 in Non-Anaesthetized Rats

Male Wistar rats (220-250 g) were tested in pairs (n=2 pairs). The animals were injected with either saline or Ro25-6981 (10.0 mg/kg, i.p.), 30 min before a tail-vein injection of [¹¹C]HC-242 (241.5±120.1 MBq at t₀). 15 min after radioactivity injection, rats were killed by decapitation. The brains were removed and homogenized in 40 volumes (v/w) of 5 mM Tris-HCl buffer (pH 7.4), using a T18 Ultra Turrax homogenizer (Ike, Staufen, Germany). 4×1 ml aliquots of the brain homogenate were immediately filtered through Whatman GF/B filters, presoaked in ice-cold buffer, using a 48-sample Brandel harvester (Alpha Biotech, Glasgow, UK). The filters were washed twice with 3 ml of ice-cold 5 mM Tris-HCl buffer, and trapped radioactivity was quantified in a 1282 Compugamma CS counter, using 5×10 μl aliquots of the injected formulation as standard. Data are expressed as the percentage of injected dose/g original wet weight tissue (% ID/g), and were compared using two-tailed Student's t-tests.

For autoradiography, pairs of male Wistar rats were injected with either saline or Ro25-6981 (20.0 mg/kg, i.p.), 30 min before a tail-vein injection of [¹¹C]HC-242 (464.3±167.5 MBq at t₀; n=2 pairs). Rats were killed by decapitation 15 min after radioactivity injection, and their brains were removed and snap-frozen in liquid nitrogen for quantitative autoradiography. 20 μm thick coronal brain sections were collected at −21° C. using a Leica CM3050S cryostat, and were immediately opposed to phosphor storage screens. The screens were read in a Storm™ 860 Phosphorimager on the following day, and the images obtained were analyzed using ImageQuant TL 8.1 software (GE Healthcare Europe GmbH, Diegem, Belgium). Data are expressed as the mean±SEM average pixel intensity of 2 rats/group, and analyzed using two-way ANOVA for the factors treatment and brain region.

### Blocking Results in Non-Anaesthetized Rats

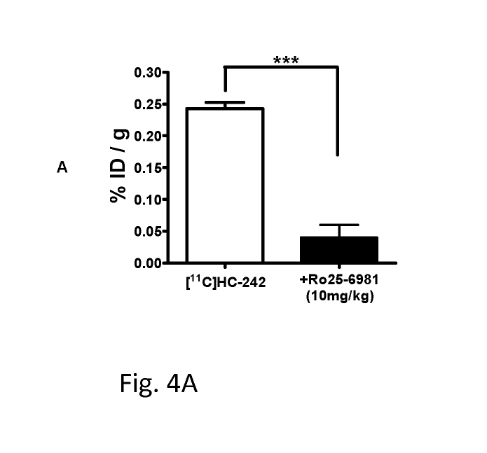

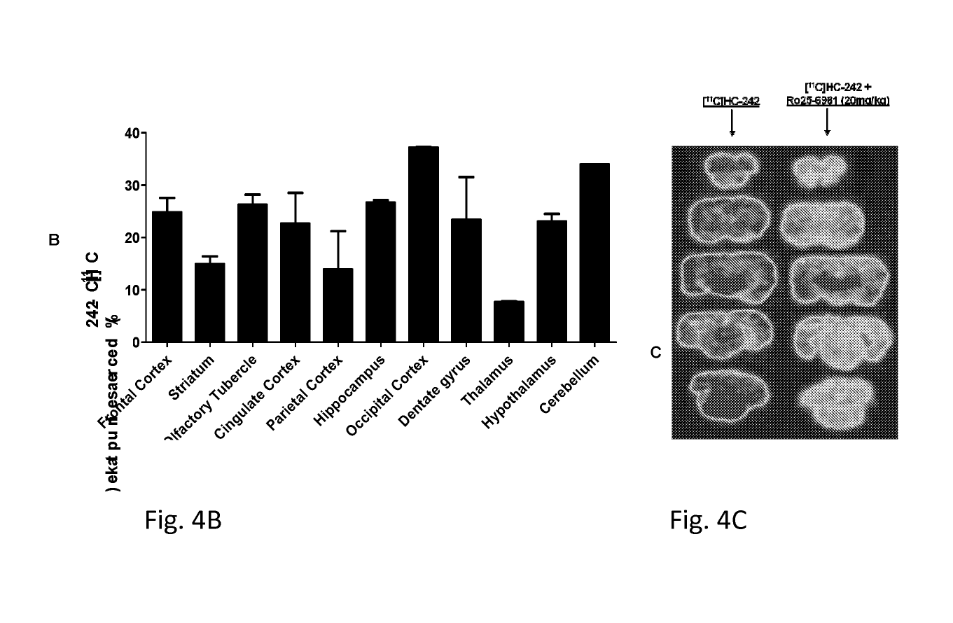

The results are presented in FIG. 4. FIG. 4 shows the quantification of [¹¹C]HC-242 uptake on brain homogenates (FIG. 4A) and sections (FIG. 4B) of control and Ro25-6981 treated rats. FIG. 4C shows representative ex vivo autoradiograms of [¹¹C]HC-242 uptake.

Pre-treatment with Ro25-6981 resulted in a significant decrease of [¹¹C]HC-242 uptake, both in brain homogenate preparations (FIG. 4A) (P<0.001, Unpaired Student's t-test) and on rat brain sections (FIGS. 4B&C) (treatment: [F_{(1,20)}=9.6, P<0.01, Two-way ANOVA). The percentage decrease in [¹¹C]HC-242 uptake compared to control was high in the occipital cortex (37.2%) and the cerebellum (33.9%), moderate in the frontal (24.8%), cingulate cortex (22.7%), hippocampus (26.7%) and the dentate gyrus (23.4%), and low in the striatum (14.9%) and thalamus (7.7%). High levels of non-specific uptake were observed in the thalamus.

### EXAMPLE 20

### Selectivity Screening Against 79 Biological Targets

HC-242 was screened against 79 biological targets by the company CEREP (France) according to their standard operating procedures (materials and methods).

### Screening Results

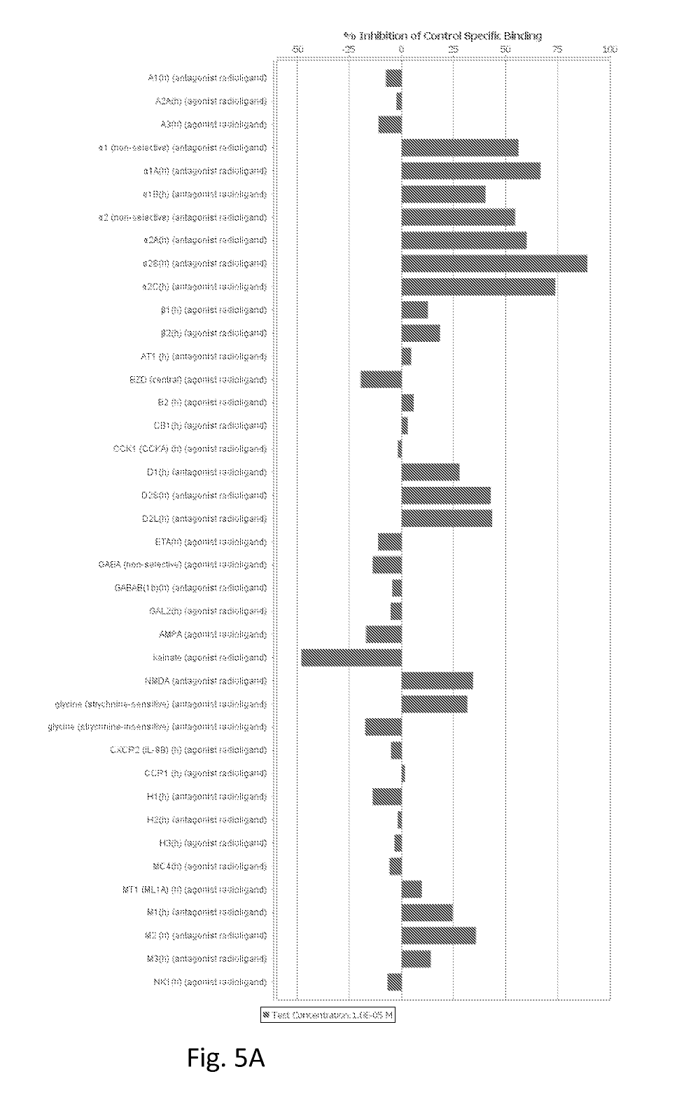

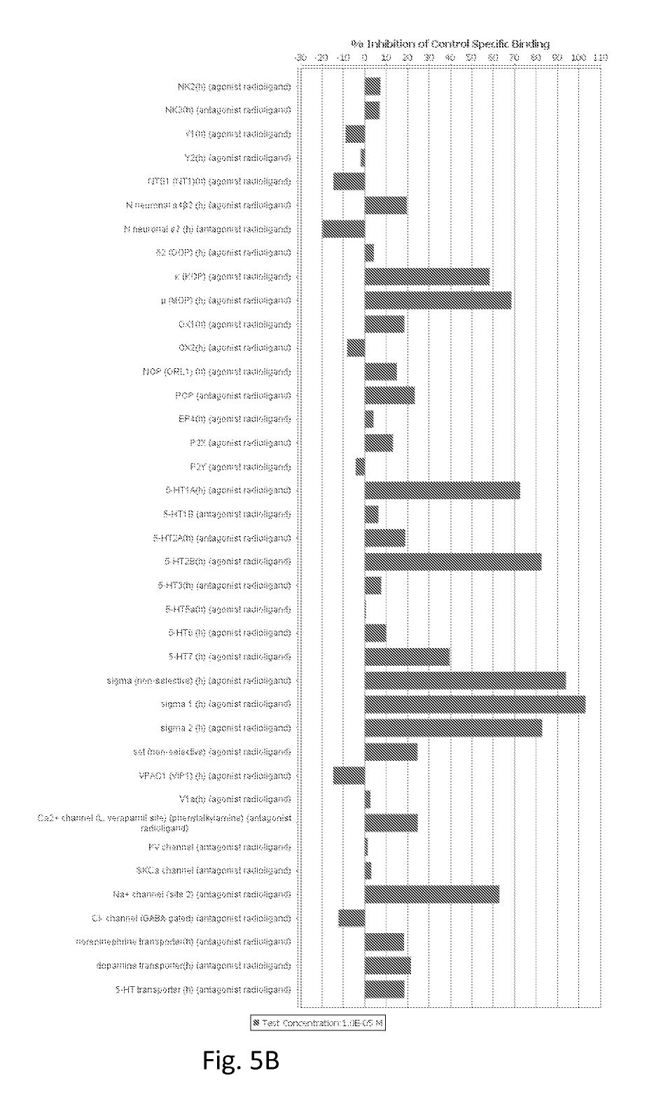

A single concentration of compound from example 1 (10 μM) did not displace 50% of specific binding for the majority of the targets tested as shown in FIGS. 5A and 5B which shows a table for the selectivity screen results for HC-242.

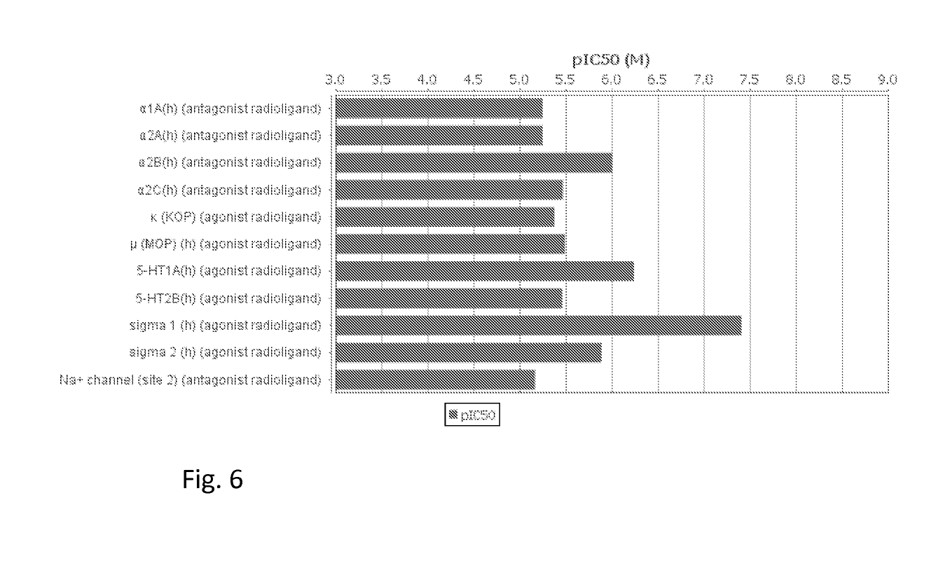

For those targets that showed >50% inhibition, exact Ki values (full dose-response inhibition curves) were determined Table 3 and FIG. 6. FIG. 6 shows the pIC₅₀ values of HC-242 for targets showing >50% inhibition in the selectivity screen assays.

TABLE 3
Affinity of HC-242 for targets showing >50%
inhibition in the selectivity screen assays.

| Target | IC50 (M) | Ki (M) | nH |
|---|---|---|---|
| alpha 1A (h) (antagonist radioligand) | 5.6E−06 | 2.8E−06 | 1.0 |
| alpha 2A (h) (antagonist radioligand) | 5.6E−06 | 2.5E−06 | 0.8 |
| alpha 2B (h) (antagonist radioligand) | 9.8E−07 | 6.5E−07 | 0.7 |
| alpha 2C (h) (antagonist radioligand) | 3.4E−06 | 1.1E−06 | 1.2 |
| kappa (KOP) (agonist radioligand) | 4.2E−06 | 2.8E−06 | 0.9 |
| mu (MOP) (h) (agonist radioligand) | 3.2E−06 | 1.3E−06 | 0.8 |
| 5-HT1A (h) (agonist radioligand) | 5.8E−07 | 3.6E−07 | 0.6 |
| 5-HT2B (h) (agonist radioligand) | 3.5E−06 | 1.7E−06 | 0.8 |
| sigma 1 (h) (agonist radioligand) | 3.9E−08 | 2.0E−08 | 0.9 |
| sigma 2 (h) (agonist radioligand) | 1.3E−06 | 9.9E−07 | 0.8 |
| Na+ channel (site 2) (antagonist radioligand) | 6.7E−06 | 6.1E−06 | 1.1 |

### EXAMPLE 21

### Determination of Log D_{oct},7.4

The distribution of the radio labelled compounds between 1-octanol and 0.2M phosphate buffer (pH=7.4) was measured in triplicate at room temperature. Briefly, 1 mL of a 20 MBq/mL solution of the radio labelled compound in 0.2M phosphate buffer (pH=7.4) was vigorously mixed with 1 mL of 1-octanol for 1 min at room temperature using a vortex. After a settling period of 30 min, five samples of 100 μL were taken from both layers. For determining recovery, 5 samples of 100 μL were taken from the 20 MBq/mL solution. All samples were counted for radioactivity. The Log D_{oct},7.4 value was calculated according to Log D_{oct},7.4=¹⁰Log(A_{oct}/A_{buffer}), where A_{oct} and A_{buffer} represent the average radioactivity counted of the 5 1-octanol and 5 buffer samples, respectively.

### Result

[¹¹C]HC-242 Log D_{oct},7.4 value=2.14±0.02

1. A compound having the structure of formula I or formula II: 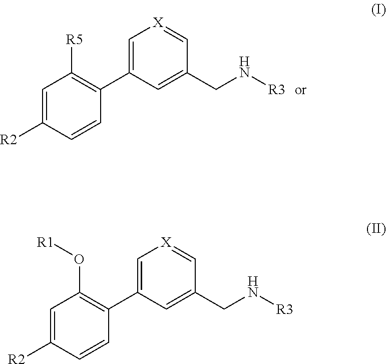
wherein R¹ is a hydrogen, or an optionally fluorinated alkoxy group having 1 to 5 carbon atoms, R² is fluorine, R5 is selected from a hydroxyl, a hydrogen or an optionally fluorinated alkoxy group having 1 to 5 carbon atoms, X is nitrogen or carbon and R³ is an organic group, or salts or solvates thereof.

2.-26. (canceled)

27. The compound according to claim 1, wherein a compound of formula (I) or (II) has the following structure: 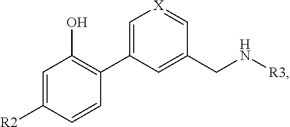
wherein R² is fluorine, X is nitrogen or carbon and R³ is an organic group.

28. The compound according to claim 26, wherein R³ is a branched or unbranched C-₁-C₆ alkyl, C₃-C₇ cycloalkyl or a methyl C₃-C₇ cycloalkyl.

29. The compound according to claim 28, wherein R³ is —CH₂-cyclopropyl, cyclobutyl or cyclopentyl.

30. The compound according to claim 27, wherein X is nitrogen.

31. A compound according to claim 1, wherein the compound is a radio labelled compound according to the following formula: 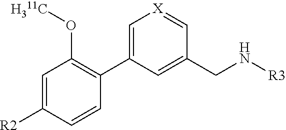
wherein the radiolabelled compound is produced by alkylation reaction of a compound of claim 27 with a [¹¹C]CH₃L compound, wherein L is a leaving group.

32. A compound according to claim 1, wherein the compound is a radio labelled compound according to the following formula: 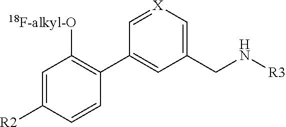
wherein the radiolabelled compound is produced by alkylation reaction of a compound of claim 27 with a ¹⁸F-alkyl-A compound, wherein A is a leaving group.

33. A compound or a salt or solvate thereof according to the following formula: 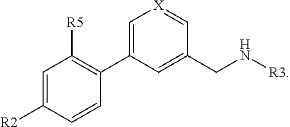
wherein X is nitrogen or carbon, R⁵ is hydrogen or an optionally fluorinated alkoxy group having 1 to 5 carbon atoms, R² is fluorine and R³ is an organic group.

34. The compound according to claim 33, wherein R³ is —CH₂-cyclopropyl, cyclobutyl or cyclopentyl.

35. The compound of claim 33, wherein R⁵ is a methoxy-alkyl group comprising a ¹¹C or a ¹⁸F isotope.

36. The compound according to claim 33, wherein R⁵ is methoxy and R³ is —CH₂-cyclopropyl.

37. The compound according to claim 33, wherein X is nitrogen.

38. The compound of claim 1, wherein the compound is radiolabelled and comprises at least one of:
X is nitrogen,
R² is fluorine,
R³ is —CH₂-cyclopropyl, cyclobutyl or cyclopentyl, and
R⁵ is a methyoxy, a methoxy-alkyl group comprising a ¹¹C or a ¹⁸F isotope.

39. The radiolabelled compound according to claim 38, wherein at least one of:
a) the carbon of the methoxy group R⁵ is a ¹¹C isotope; or
b) the optionally fluorinated alkoxy group R⁵ is an alkoxy group having from one to five fluorine substituents; or
c) R⁵ is hydrogen and R² is a radio labelled ¹⁸F atom.

40. The radiolabelled compound according to claim 39, wherein the optionally fluorinated alkoxy group R⁵ is substituted with one fluorine atom and wherein the fluorine atom is a radio labelled ¹⁸F atom.

41. A method for the in vivo diagnosis or imaging of a NMDA related disease in a subject, preferably a human, comprising administration of a radiolabelled according to claim 38.
